(19) United States
(12) Patent Application Publication
(10) Pub. No.: US 2004/0230173 A1
(43) Pub. Date: Nov. 18, 2004

(54) Liquid acquisition layer with caliper recovery and the method for producing the same

(76) Inventors: Patrick Barge, Cornelius, NC (US); Mark Landreth, Mooresville, NC (US); Nick M. Carter, Mooresville, NC (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/739,421

(22) Filed: Dec. 18, 2003

Related U.S. Application Data
(60) Provisional application No. 60/434,909, filed on Dec. 20, 2002.
(60) Provisional application No. 60/435,092, filed on Dec. 20, 2002.

(51) Int. Cl. A61F 13/15; A61F 13/20

(52) U.S. Cl. 604/367; 604/385.01

(57) ABSTRACT

The present invention is directed to a nonwoven fabric for hygienic articles for absorbing liquids, such as diapers, or other sanitary articles that comprise a liquid acquisition layer positioned between the liquid permeable outer layer and the absorbent core, and more specifically, the present invention relates to a liquid acquisition layer with improved caliper recovery and re-wet properties.

### TECHNICAL FIELD

The present invention generally relates to a nonwoven fabric for hygienic articles for absorbing liquids, such as diapers, or other sanitary articles that comprise a liquid acquisition layer positioned between the soft porous outer layer and the absorbent core, and more specifically, the present invention relates to a liquid acquisition layer with improved caliper recovery and re-wet properties.

### BACKGROUND OF THE INVENTION

Modem disposable absorbent articles such as diapers and other sanitary articles generally comprise an absorbent core made from cellulose pulp or other organic fibers and/or a superabsorbing polymer in powder or fiber form. The portion of the core which faces the body is covered with a soft nonwoven fabric or perforated film. In addition, it is common to provide an additional layer of nonwoven fabric between the outer fabric layer and the core. The purpose of this layer, often referred as a liquid transport or surge layer, is to accommodate a surge of liquid and to allow more time for the core to absorb the liquid without overflow and leaking. A second function of the intermediate layer is to isolate the core from the outer layer fabric or top sheet and to minimize re-wetting of the outer layer.

Many proposals have been made with regard to liquid acquisition layers. Generally, the use of bulky fabrics having a high void volume and high wet compression resistance have been proposed, with the high void volume providing transient liquid handling capacity, and the compression resistance improving re-wet values. Many of these proposals may be considered as deficient, inasmuch as a surge of liquid is allowed to pass through and concentrate on one area of the core, thereby reducing efficiency of absorption. Also, these fabrics tend to increase the overall bulkiness of the article.

In order to keep bodily fluids and excretions away from the skin, absorbent articles such as diapers, sanitary napkins, incontinence pads and the like, tend to be multi-layered consisting of a liquid permeable cover layer, a fluid acquisition layer, which is also known as a transfer layer, and an absorbent containment layer. Manufacturers of such absorbent articles, require ways to improve the comfort of these layers while also improving the functionality and aesthetic qualities.

An important comfort feature of an absorbent article is its ability to retain a feeling of dryness against the user's skin after use. To preserve a dry feeling on a liquid permeable cover layer, a transfer layer must manage the acquisition of fluid from the cover layer through and into the absorbent containment layer in a timely and effective manner. The transfer layer also provides an aesthetic purpose in that it essentially obscures the absorbent containment layer and softens the edges of the absorbent core.

A resilient, hydrophilic acquisition layer is highly beneficial to the overall performance of the absorbent article. The acquisition layer actively channels fluid away from the skin and sequesters it into an underlying absorbent core. As the acquisition layer is sandwiched in between two outer layers, the acquisition layer has a tendency to become crushed and deformed during use. Deformation of the transfer layer compromises the proper channeling of fluid away from the body and can ultimately lead to critical article failure such as leaking of fluid.

It is an object of this invention to make available a nonwoven fabric with improved re-wet values and improved caliper recovery for use in absorbent articles.

### SUMMARY OF THE INVENTION

The present invention is directed to a nonwoven fabric for hygienic articles for absorbing liquids, such as diapers, or other sanitary articles that comprise a liquid acquisition layer positioned between the liquid permeable outer layer and the absorbent core, and more specifically, the present invention relates to a liquid acquisition layer with improved caliper recovery and re-wet properties.

In accordance with the present invention, an absorbent article is provided with an inner absorbent core and an outer layer, together with a liquid acquisition layer between the core and outer layer. The acquisition layer is a nonwoven bonded with an adhesive blend which incorporates a modified high carboxylation (high acid) binder. The acquisition layer operates to provide a one-way valve effect toward the core and improved re-wet properties. Further, the acquisition layer has an improved caliper recovery after compression with use of the modified high carboxylation binder.

The nonwoven fabric of the liquid acquisition layer is a single or multi-layered fabric, with the fibers and/or filaments being bonded together by a modified high carboxylation (high acid) binder. Absorbent articles can be furnished with the acquisition layer of the present invention for better performance attributes. Utilization of the binder blend with the modified high carboxylation (high acid) binder results in significantly higher wet tensile, superior liquid handling characteristics, and improved re-wet properties versus a standard high carboxylation binder type. Further, with the use of this binder blend, the acquisition layer has improved re-bulk properties after compression at elevated temperatures, such as of or about 45° C. Further still, the acquisition layer with use of this binder blend has an improved balance of re-wet and of bulk recovery properties after compression at elevated temperatures.

### BRIEF DESCRIPTION OF THE DRAWINGS

FIG. 1 is an enlarged schematic cross sectional fragmentary view of the absorbent article of the present invention;

FIG. 2 shows a bar chart and data table illustrating improved caliper with a modified binder blend in accordance with the present invention;

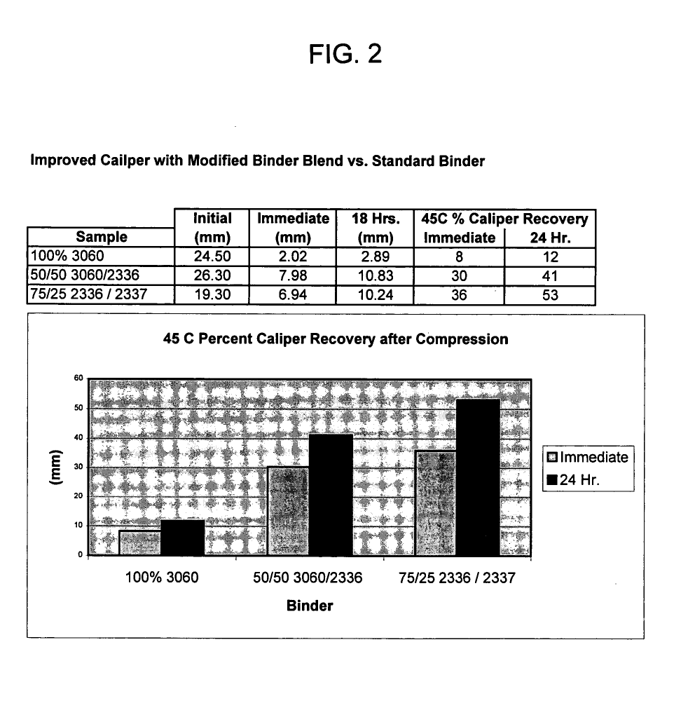

FIG. 3 is a graph and accompanying data table showing improved re-wet with the modified binder blend in accordance with the present invention; and

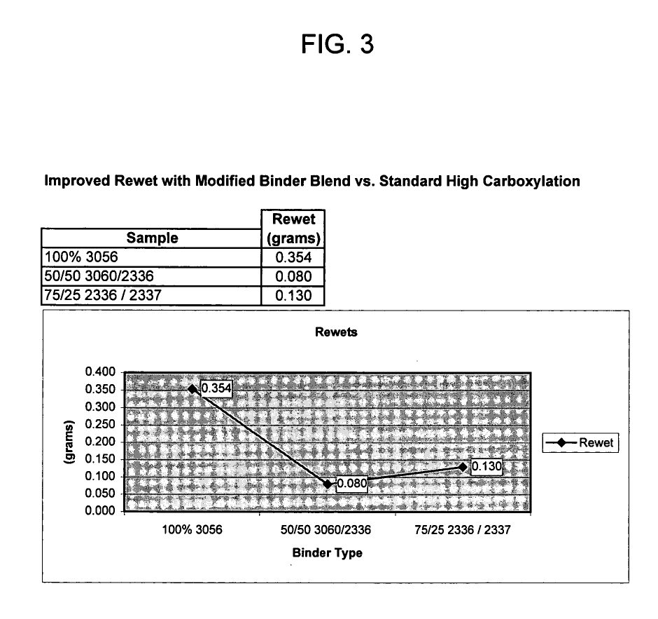

FIG. 4 is a bar chart and data table showing improved machine-direction tensile strengths achieved with the modified binder blend in accordance with the present invention.

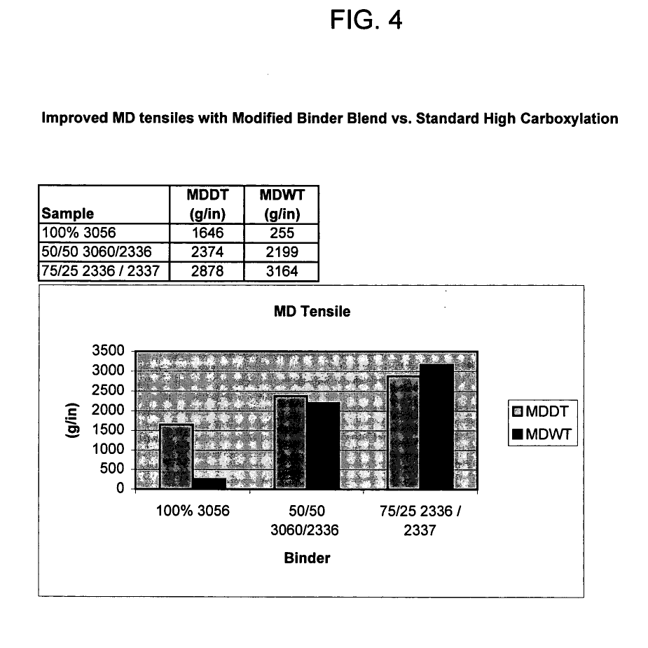

### DETAILED DESCRIPTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

The liquid acquisition layer of the present invention is provided in the form of a nonwoven fabric comprised of a binder blend, which includes a modified high carboxylation (high acid) binder, and provides an absorbent article with superior liquid handling characteristics, as well as improved re-wet properties. The acquisition layer is a nonwoven web, wherein the web may include one or more layers of continuous filament webs, carded staple length fiber webs, or a combination thereof. In addition, the resultant web may be treated with one or more various mechanically altering techniques, such as compaction, or treated with one or more performance altering additives.

Various types of fibers may be used to make the acquisition layer of the present invention, which can include the use of staple length fibers, continuous filaments, and the blends of fibers and/or filaments having the same or different composition. Fibers and/or filaments are selected from synthetic compositions of homogeneous or mixed fiber length. Synthetic fibers, which may be blended in whole or part, include thermoplastic and thermoset polymers. Thermoplastic polymers suitable for blending with dispersant thermoplastic resins include polyolefins, polyamides and polyesters. The thermoplastic polymers may be further selected from homopolymers; copolymers, conjugates and other derivatives including those thermoplastic polymers having incorporated melt additives or surface-active agents. Staple lengths are selected in the range of 0.25 inch to 10 inches, the range of 1 to 3 inches being preferred and the fiber denier selected in the range of 1 to 22, the range of 5.0 to 10 denier being preferred for general applications.

The fabric of the acquisition layer may be formed into a nonwoven web by means of various technologies or a combination of differing technologies, examples include, but are not limited to carded webs.

The present invention contemplates a multiple layer liquid acquisition fabric having two or more layers bonded together. Preferably, the layers comprise fibers which are compatible in the sense that the layers are capable of thermally self-bonding to each other. In addition, the fibers in the acquisition layer are preferably hydrophilic or treated with a surface acting agent that imparts the fabric with increased wettability as compared to the outer fabric.

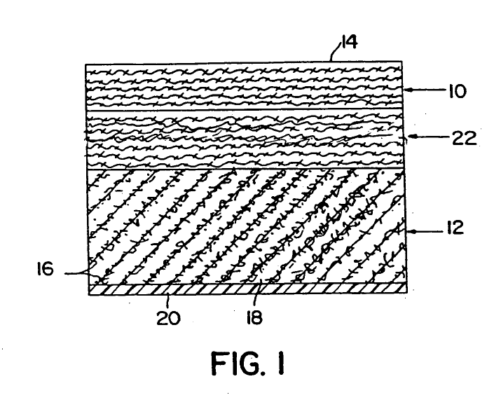

The acquisition layer of the present invention is suitable for use in various absorbent articles including diapers, incontinence articles, and feminine hygiene products. As shown in FIG. 1, the absorbent article of the present invention comprises an outer nonwoven soft porous outer layer 10 and an inner absorbing core 12. Outer layer 10 is a nonwoven fabric of polymer fibers bonded together to provide a porous, planar web. The fabric, for example, can be made from relatively fine (less than 3.0 dpf) polyolefin fibers or bi-components fibers which are thermally point bonded together, such as by calendering between engraved calender rolls, or through-air bonding with hot air, or by other processes, well known in the art of nonwovens. As the fabric is in contact with the body, the outwardly facing surface 14 should have a soft feel or surface, and the fabric should be porous to allow rapid transfer of liquids. In some situations, it may be possible to substitute an apertured film for the layer 10, or an apertured film which is covered by fibers which are either adhesively or thermally bonded to the film. In the case of conventional point bonded nonwoven fabrics made from polyolefin, especially polypropylene, the basis weight of the fabric will be in the order of from about 15 to about 30 gsm.

The core 12 is a relatively thick and highly absorbent structure designed to permanently absorb and retain repeated insults of liquids. Cores, such as used in disposable diapers, are well known in the art and typically comprise unbonded cellulose pulp fibers, with a quantity of superabsorbent polymer (SAP) 16 disposed therein in particle or fiber form. Other variants include cores without SAP particles, as well as cores in which the SAP is the primary or only absorption media. The core is typically provided in a flat rectangular or hourglass shape, depending on the size, fit and other practical parameters of the disposable garment or other article.

In many cases, the surface 18 of the core 12 remote from the outer porous fabric 10 is provided with a layer 20 of a liquid impermeable material, such as an impermeable, or semi-permeable (breathable) film. The outer layer of the film 20 is sometimes covered with a layer of fibers or is otherwise treated to simulate a cloth-like fabric.

The foregoing descriptions of the outer layer 10, absorbent layer 12 and outer layer 20 are intended as general descriptions of conventional components used in present day disposable sanitary or absorbent articles and garments, and many variants are known and are described in the prior art. In many cases, the core 12 will be substantially thicker than that shown in FIG. 1, and the thickness of the fabric and film layers has been exaggerated for sake of clarity.

In accordance with the present invention, a liquid acquisition layer 22 of a particular configuration is disposed between the outer layer 10 and the core 12 and in surface-wise contact with the outer and core. The acquisition layer 22 comprises two or more layers bonded together with a binder blend including one or more modified high carboxylation (high acid) binders. In a first embodiment, the binder mixture comprised a high carboxylation blend of about 50% 401-054 (modified 3056, also known as 2336) and about 50% 3060. The 401-054 (variation of 3056 binder) is a modified high carboxylated (high acid) binder, and both binders are identifiable and available through Omnova Solutions. In a second embodiment, the binder mixture comprised a modified high carboxylation blend of about 50% 401-054 (modified 3056, also known as 2336) and about 50% 401-049 (also known as 2337). Both 401-054 (2336) and 401-049 (2337) are high carboxylated (high acid) binders. Again, the aforementioned binders are identifiable and available through Omnova Solutions. The aforementioned binder compositions may be applied by various techniques known in the art, such as spraying, padding, kiss coating, and immersion coating.

Catamenial products, such as feminine hygiene pads, are of the same general construction as the aforementioned diaper structure. Again, a topsheet and a backsheet are affixed about a central absorbent core. The overall design of the catamenial product is altered to best conform to the human shape and for absorbing human exudates. Representative prior art to such article fabrication include U.S. Pat. No. 4,029,101, U.S. Pat. No. 4,184,498, U.S. Pat. No. 4,195,634, and U.S. Pat. No. 4,886,513, which are incorporated herein by reference.

The nonwoven fabric of the acquisition layer of the present invention comprising the high carboxylation binders were tested for re-wet and caliper recovery. Re-wet testing was performed in accordance with the EDANA #151.1-96 test method. Caliper recovery was determined via a compression test, wherein ten plies of fabric were placed between two plexiglass plates and compressed on a Carver Press to a thickness of approximately 2.3 mm. The plies were compressed for a predetermined time period, pressure was released after such time period, the sample was removed, and immediately measured for caliper. Caliper is re-measured after 1 hour, 5 hours, and 24 hours. The % caliper recovery is calculated by the following formula:

24 hr. relaxed caliper×100/Initial Caliper

Table 1 comprises tensile and re-wet results for samples made in accordance with the present invention. Table 2 shows the compression test results for three samples also made in accordance with the present invention. As noted in the Table, Sample A (10 plies of material) was compressed to approximately 2.3 mm at a temperature of 45° C . The initial caliper was 26.3 mm for 10 plies. After 24 hrs. Sample A had a caliper of 10.83 mm, which demonstrates a recovery of or about 41%. Sample B which is a blend of2 modified high carboxylation binders was compressed to 2.3 mm (for 10 plies) at 45°. The initial caliper before compression was 19.3 mm for 10 plies. After removing compression and sample relaxed for 24 hrs, the caliper returned to 10.24 mm, or recovered 53%. The acquisition layer of the present invention preferably has a recovery of at least 40%, more preferably 45%, and most preferably a recovery of at least 50% after 24 hours.

From the foregoing, numerous modifications and variations can be effected without departing from the true spirit and scope of the novel concept of the present invention. It is to be understood that no limitation with respect to the specific embodiments disclosed herein is intended or should be inferred. The disclosure is intended to outer, by the appended claims, all such modifications as fall within the scope of the claims.

TABLE 1

| | | | | | MDT | MDWT | Re-wet |
|---|---|---|---|---|---|---|---|
| | | | | | ASTM | ASTM | EDANA |
| | | | Weight | Caliper | D-5035 | D-5035 | #151.1-96 |
| Sample | Ratio | Binder | (gsm) | (mm) | (g/m) | (g/m) | (gm) |
| A | 50/50 | 401-054/3060 | 59.8 | 2.63 | 23/4 | 2199 | 0.08 |
| B | 75/25 | 2336/2337 | 60.9 | 1.93 | 28/8 | 3164 | 0.13 |

TABLE 2

| | | | | | | | | Caliper | Caliper |
|---|---|---|---|---|---|---|---|---|---|
| | | | Compression Test (mm) @ 113F | | | | | Recovery | Recovery |
| Sample | Ratio | Binder | Initial | Immediate | 1 hr. | 5 hr. | 24 hr. | Immediate | 24 hr. |
| C | 50/50 | 401-054/3060 | 26.3 | 7.98 | 8.75 | 9.28 | 10.83 | 30 | 41 |
| D | 75/25 | 2336/2337 | 19.3 | 6.94 | 8.16 | 9.05 | 10.24 | 36 | 53 |

What is claimed is:

1. A nonwoven acquisition layer for an absorbent article with a caliper recovery performance of at least 40% after 24 hours comprising at least one modified high carboxylation binder.

2. A nonwoven acquisition layer as in claim 1, wherein said acquisition layer is more than one layer.

3. An absorbent article comprising:
a. a liquid pervious top sheet;
b. a liquid impervious backsheet;
c. an absorbent core position between said top sheet and said backsheet; and
d. a liquid acquisition layer positioned between said top sheet and said absorbent core wherein said liquid acquisition layer is comprised of at least one modified high carboxylation binder and has a caliper recovery performance of at least 40% after 24 hours.

4. A method of making a nonwoven fabric with at least 40% caliper recovery after 24 hours comprising the steps of:
a. providing a fibrous batt;
b. providing a modified high carboxylation binder; and
c. coating said fibrous batt with said modified high carboxylation binder.

5. A method of making a nonwoven fabric with at least 40% caliper recovery after 24 hours as in claim 4, wherein said nonwoven fabric is a liquid acquisition layer.
